(19) United States
(12) Patent Application Publication
(10) Pub. No.: US 2020/0211715 A1
(43) Pub. Date: Jul. 2, 2020

(54) METHOD FOR RECRUITING PATIENTS, AND PATIENT RECRUITMENT SYSTEM

(71) Applicant: Clinerion Ltd., Basel (CH)

(72) Inventors: Andreas WALTER, Baden-Baden (DE); Dominik ARONSKY, Rueschlikon (CH); Ulf CLAESSON, Wettswil (CH)

(21) Appl. No.: 16/620,112

(22) PCT Filed: Jun. 8, 2017
(86) PCT No.: PCT/EP2017/064024

§ 371 Date: Dec. 6, 2019

(51) Int. Cl. G16H 50/70; G16H 50/30; G16H 10/60; H04W 4/029

(52) U.S. Cl. CPC G16H 50/70; H04W 4/029; G16H 10/60; G16H 50/30

(57) ABSTRACT

A method for recruiting patients for a study, in particular for at least one clinical study, includes at least one matching step that involves at least one patient parameter of at least one patient, stored on a patient data server, being matched, in a computerized manner, with at least one recruitment parameter of at least one study, stored on a study data server, and at least one rating step that involves at least one suitability parameter of a suitability of the patient for the study being determined, in a computerized manner, on the basis of the patient parameter and the recruitment parameter and stored on at least one server, in particular a patient recruitment server. In at least one association step at least an association between the patient and the study is made on the basis of the suitability parameter, and at least one piece of association information is stored on at least one server, in particular the patient recruitment server.

### PRIOR ART

The invention relates to a method for recruiting patients according to the preamble of claim 1 and to a patient recruitment system according to the preamble of claim 19.

The prior art discloses patient databases in which patient data records are storable that can be used for recruiting patients for clinical studies.

The object of the invention is in particular to attain advantageous properties for a patient recruitment. Moreover, an object of the invention is in particular to attain a high level of quality and/or efficiency. Additionally, an object of the invention is in particular to attain selection of possible participants for clinical studies with a high level of quality. The object is achieved according to the invention by the features of claims 1 and 19, while advantageous configurations and developments of the invention can be found in the subclaims.

### ADVANTAGES OF THE INVENTION

The invention is based on a method, in particular a patient recruitment method, for recruiting patients for a study, in particular for at least one clinical study, wherein at least one matching step involves at least one patient parameter of at least one patient, stored on a patient data server, being matched in a computerized manner, and in particular in automated fashion, with at least one recruitment parameter of at least one study, stored on a study data server, and wherein at least one rating step involves at least one suitability parameter of a suitability of the patient for the study being determined in a computerized manner, and in particular in an automated fashion, on the basis of the patient parameter and the recruitment parameter and stored on at least one server, in particular a patient recruitment server.

It is proposed that in at least one association step at least an association between the patient and the study is made on the basis of the suitability parameter, and at least one piece of association information is stored on at least one server, in particular the patient recruitment server.

The method according to the invention allows advantageous properties for a patient recruitment, in particular for the efficiency and/or quality thereof, to be attained. Advantageously, a high level of quality for a selection of suitable patients can be attained. Additionally, suitable patients can advantageously be encouraged to participate in clinical studies and/or a level of recruitment effort can be reduced. Moreover, a patient and/or a clinical personnel associated with the patient can advantageously be informed about suitable studies and prompted into active behavior. Further, an awareness of clinical studies by patients can be produced and this allows an effort for patient recruitment to be reduced. In addition, a high level of cost efficiency can be attained for a patient recruitment. In particular, patients can be efficiently recruited for studies in connection with proof of an effectiveness of active substances and/or methods of treatment, in particular those to be licensed in future.

Moreover, the invention comprises a patient recruitment system, in particular for recruiting patients for studies, advantageously for clinical studies, in particular configured to perform the method for recruiting patients, having at least one patient recruitment server that is configured to carry out at least one matching process for matching at least one patient parameter of at least one patient with at least one recruitment parameter of at least one study and to carry out at least one rating process for generating at least one suitability parameter of a suitability of the patient for the study on the basis of the patient parameter and the recruitment parameter.

It is proposed that the patient recruitment server is configured to carry out at least one association process for making at least one association between the study and the patient on the basis of the suitability parameter.

The patient recruitment system configuration according to the invention allows advantageous properties for a patient recruitment, in particular for the efficiency and/or quality thereof, to be attained. Advantageously, a high level of quality for a selection of suitable patients can be attained. Additionally, suitable patients can advantageously be encouraged to participate in clinical studies and/or a level of recruitment effort can be reduced. Moreover, a patient can advantageously be informed about suitable studies and prompted into active behavior. Further, an awareness of clinical studies by patients can be produced and this allows of a level of effort for a patient recruitment to be reduced. In addition, a high level of cost efficiency can be attained for a patient recruitment.

In particular, the matching process involves, in particular computerized, performance of the matching step and/or the rating process involves, in particular computerized, performance of the rating step and/or the association process involves, in particular computerized, performance of the association step. In this context, a “process” is supposed to be understood to mean in particular a computerized routine and/or computerized subroutine or program execution that is performable in particular by means of at least one computing unit and/or is performed in at least one operating state. A “computing unit” is supposed to be understood to mean in particular a unit having an information input, information processing and an information output. Advantageously, the computing unit has at least one processor, a memory, one or more input and output means, further electrical components, an operating program, closed-loop control routines, open-loop control routines and/or computing routines.

Preferably, the method is configured for recruiting patients. Particularly preferably, the method and/or the patient recruitment system is configured to recruit patients for phase III clinical studies. In addition, recruitment of patients for another phase of clinical studies and/or for other types of studies is also conceivable, however. In particular, the patient recruitment can comprise a recruitment of patients in the medical and/or veterinary sector, advantageously for applicable, in particular clinical, studies. “Configured” is supposed to be understood to mean in particular specifically programmed, designed and/or equipped. An object being configured for a specific function is supposed to be understood to mean in particular that the object performs and/or carries out this specific function in at least one application state and/or operating state. A method being “configured” for a purpose is supposed to be understood to mean in particular that the method involves at least one method step that is specifically aimed at the purpose and/or that the method is specifically directed to the purpose and/or that the method is used to fulfill the purpose and/or is at least partly optimized to fulfill it. A method step being “configured” for a purpose is supposed to be understood to mean in particular that the method step is aimed specifically at the purpose and/or that the method step is specifically directed to the purpose and/or that the method step is used to fulfill the purpose and/or is at least partly optimized to fulfill it.

A “patient data server” is supposed to be understood to mean in particular a server on which patient data records are storable and/or stored. The patient data server can be in the form of a dedicated server, but alternatively or additionally also in the form of a virtual server and/or a cloud server and/or a shared server and/or in the form of at least part of a content delivery network or another suitable server. Preferably, the patient parameter is at least part of at least one, in particular electronic, patient data record. In particular, the patient data record can comprise a plurality of different patient parameters. The patient parameter comprises in particular at least one value and/or at least one characteristic quantity that denotes at least one feature of the patient and in particular the state of health of said patient and/or the body of said patient, for example at least one age, in particular a minimum age and/or a maximum age, a height, in particular a minimum height and/or a maximum height, a weight, in particular a minimum weight and/or a maximum weight, a BMI, in particular a minimum BMI and/or a maximum BMI, a sex, a medical history, a laboratory value, a measured value, medication information, a sensor value, addiction information, habit information, behavioral information and/or the like, with any combinations being conceivable. It is also conceivable for the patient parameter and/or the patient data record to comprise at least one, in particular unique, patient ID. In particular, the patient data record comprises at least one case comprising at least one diagnosis and/or at least one piece of treatment information and/or at least one piece of medication information linked to the treatment information and/or at least one progression of a disease or the like. In particular, the patient data record comprises a plurality of such cases and advantageously at least part of a treatment and/or medical history of the patient.

A “study data server” is supposed to be understood to mean in particular a server on which study data records, in particular data records pertaining to clinical studies, are storable and/or stored. The study data server may be in the form of a dedicated server, but alternatively or additionally also in the form of a virtual server and/or a cloud server and/or a shared server and/or in the form of at least part of a content delivery network or another suitable server. Preferably, the recruitment parameter is at least part of at least one, in particular electronic, study data record, in particular of a data record for a clinical study. In particular, the study data record can be a data record for a clinical study. By way of example, the study data record can comprise at least one piece of contact information, for example a person responsible for the study, and/or at least one piece of information about a study period and/or a study location and/or a study type and/or a study aim or other suitable metadata. Preferably, the recruitment parameter comprises at least one value and/or at least one value range and/or at least one characteristic quantity and/or at least one piece of categorization information, in particular a plurality of concatenated pieces of categorization information, and/or at least one head word, in particular a, for example even hierarchic combination of head words, on the basis of which a suitability of a patient, in particular on consideration of the patient parameter, is determinable, preferably in a computerized manner. By way of example, the recruitment parameter can comprise a sex, an age, a height, a weight, a BMI, a medical history, a laboratory value, a measured value, applicable minimum and/or maximum values, a medication, a sensor value, addiction information, habit information, behavioral information and/or the like, in particular in the form of a value range and/or in the form of a decision criterion such as for example a Boolean query and/or a categorical parameter. Moreover, it is conceivable for the study parameter and/or the study data record to comprise at least one, in particular unique, study ID.

In this instance, patient parameters and/or study parameters can fundamentally comprise for example textual information and/or at least one reference to medical and/or clinical terminologies, in particular in the form of a reference to an applicable database and/or at least one piece of image information and/or audio information and/or video information and/or at least one timestamp and/or a chronology of the like.

A “patient recruitment server” is supposed to be understood to mean in particular a server on which, in particular electronic, data for a recruitment of specific patients for specific studies are storable and/or stored. The patient recruitment server may be in the form of a dedicated server, but alternatively or additionally also in the form of a virtual server and/or a cloud server and/or a shared server and/or in the form of at least a part of a content delivery network or another suitable server. Preferably, at least one association data record and/or at least one suitability data record is stored on the patient recruitment server. It is conceivable for the suitability data record to contain a plurality of different suitability parameters. Advantageously, the suitability data record is a suitability data record associated with the patient. Particularly advantageously, the suitability parameter is a suitability parameter associated with the study and with the patient. Preferably, a plurality of different suitability data records, which are in particular each associated with a specific patient and/or which each contain suitability parameters for a specific patient for at least one study, are stored on the patient recruitment server.

In particular, the patient data server and/or the study data server and/or the patient recruitment server has at least one computing unit and/or at least one interface to a central computing unit and also preferably at least one electronic data memory for storing parameters and/or data records or the like. Preferably, parameters and/or data records stored on the data memory are encrypted. Particularly preferably, data are retrieved from the data memory and/or stored thereon merely in anonymized and/or pseudonymized form. Advantageously, the patient data server and/or the study data server and/or the patient recruitment server are at least intermittently connected by means of at least one data connection. Preferably, the patient data server and/or the study data server and/or the patient recruitment server are, at least in part, of integral and/or identical design. In particular, it is conceivable for the patient recruitment server to comprise and/or form the patient data server and/or the study data server. It is furthermore conceivable for the patient data server and/or the study data server and/or the patient recruitment server to be arranged at different locations or at the same location and/or part of a cloud. Advantageously, the patient data server and/or the study data server and/or the patient recruitment server are integrated in an Intranet of a healthcare institution, for example a hospital, a clinic, a medical center or the like, and/or connectable to an external network, in particular merely for maintenance purposes, so that a high level of security and/or anonymity for personal data can advantageously be ensured.

In particular, the matching step comprises a computerized comparison of the patient parameter with a recruitment parameter and/or at least one computerized call to and/or query of the patient parameter and of the recruitment parameter. Preferably, the patient recruitment server is configured to perform the matching step. In particular, it is conceivable for the matching step to involve at least one patient data record and/or at least one study data record being queried and/or at least temporarily stored, in particular for preparation of the rating step. Preferably, the matching step comprises a comparison of a set of patient parameters with a specific logic combination of a plurality of recruitment parameters that, for example hierarchically and/or at least in part, are used as inclusion and/or exclusion criteria for formulating the logic combination. The logic combination is advantageously produced in a computerized manner and/or used for a computerized production of at least one computerized-performable decision and/or rating process.

Preferably, the rating step comprises at least one computerized calculation and/or determination of the suitability parameter, for example in the form of a logical query, a numerical value comparison, an intersection formation or the like and preferably a combination of at least two of these. In particular, it is conceivable for the suitability parameter to be determined, in a computerized manner, on the basis of a specific set of patient parameters and/or on the basis of a specific set of recruitment parameters. Preferably, the suitability parameter is a measure of quality for a suitability of the patient for the study, in particular a match score. It is conceivable for the suitability parameter to be a Boolean parameter, for example having the two states “patient suitable” and “patient unsuitable”. Preferably, the suitability parameter is a match parameter, in particular the match score, that preferably correlates with a quality of a match between the patient and the study. By way of example, the suitability parameter can be a percentage for a suitability. Further, it is conceivable for the suitability parameter to be able to assume different discrete values, for example values between 0 and 5, between 0 and 10, between 0 and 100 or any other discrete values. Preferably, the suitability parameter is determined as a function of a plurality of patient parameters and a plurality of study parameters, with individual parameters and/or matches being able to be weighted at different levels. Moreover, it is conceivable for specific parameters to be handled as exclusion parameters. By way of example, it is conceivable for a patient to be rated as completely unsuitable if at least one patient parameter is not within a value range permitted according to an applicable recruitment parameter and/or does not correspond to a specific recruitment parameter, such as for example a sex and/or a specific medication on the basis of which a patient is for example unsuitable for an applicable study. Alternatively or additionally, it is conceivable for the suitability parameter to be dependent on a number and/or manifestation of a concordance between patient parameters and applicable recruitment parameters.

By way of example, the study can contain recruitment parameters requiring a specific sex, an age in a specific value range, an existence of a specific illness and a laboratory value of a specific health-related variable in a specific value range from a potentially suitable patient. Naturally, any other combinations that appear appropriate to a person skilled in the art are conceivable. In this case, for example the suitability parameter could assume an average value if the patient parameters for sex and existence of the illness are consistent with the recruitment parameters and the patient has an appropriate age, and could assume a high value if for example additionally a laboratory value of the patient falls into the appropriate value range. The sex could furthermore be defined as an exclusion criterion, for example, so that for example in the case of a patient with a sex which is not consistent with that required by the study, a suitability parameter indicating nonsuitability is produced, irrespective of a match for the other parameters. In this case, any combinations and strategies that appear appropriate to a person skilled in the art for determining a suitability parameter are naturally likewise conceivable and the cited examples should be understood to be completely nonlimiting.

Preferably, the association comprises linking the patient to the study, in particular while recording the suitability parameter. It is preferably possible for a plurality of studies and in particular applicable suitability parameters to be associated with a specific patient. Similarly, a plurality of patients and in particular applicable suitability parameters can be associated with a specific study. Advantageously, the method comprises at least one pseudonymization step, in which at least one patient parameter and/or at least one patient data record is pseudonymized at least in part. Similarly, anonymization is also conceivable. Fundamentally, all of the personal data of the patient are advantageously pseudonymized and/or anonymized at a suitable point in the method. In particular, in the association step the association with the patient is made in pseudonymized and/or anonymized form, and/or the association information is advantageously stored on the patient data server in pseudonymized and/or anonymized form.

In an advantageous configuration of the invention, it is proposed that the association step involves the study being associated with the patient on the basis of the suitability parameter. In particular, the study is associated with the patient if the suitability parameter indicates that the patient is suitable for the study, in particular if the suitability parameter is above or below a specific limit value. In this case, it is conceivable for the limit value to be adapted, in a computerized manner, on the basis of at least one piece of information stored on the patient recruitment server and/or on the study data server. By way of example, the limit value can be increased or decreased if a number of potentially suitable patients increases or reduces. Moreover, it is conceivable for the limit value to be presettable by clinical personnel and/or by the patient, for example. Additionally, it is conceivable for the limit value to be stored on the study data server as a recruitment parameter and/or to be presettable by an originator of and/or party responsible for the study. This advantageously allows suitability information to be linked to a patient. Advantageously, this allows suitable patients to be found easily and/or efficiently.

In a particularly advantageous configuration of the invention, it is proposed that the association step involves a plurality of suitable studies being associated with the patient. In particular, at least one respective study parameter and advantageously a respective applicable suitability parameter is associated with the patient, and an applicable association function is advantageously stored on the patient recruitment server. Preferably, a respective suitability parameter of the patient is produced for a specific selection of studies and/or for all studies for which study data are stored on the study data server, and suitable studies are advantageously then associated with the patient. In this case, studies can be selected in a computerized manner and/or on the basis of at least one pre-settable parameter. This advantageously allows a time and/or cost saving to be attained when recruiting patients for suitable studies.

It is furthermore proposed that the association step involves an ordered list of suitable studies being associated with the patient, said list being stored on at least one server, in particular the patient recruitment server. A study is suitable in this case in particular if the patient is at least partly suitable or at least not unsuitable as a test subject for the study. Moreover, it is conceivable for a further, in particular ordered, list of currently unsuitable or partially suitable studies to be produced and associated with the patient, in particular a list containing studies that are potentially suitable in future. In this case, an automated decision regarding a future potential suitability is conceivable, for example on the basis of a patients current medication and/or health situation or the like on the basis of which the patient is currently unsuitable for the study, but the end of which is foreseeable and/or after the end of which the patient would be suitable for the study. This advantageously allows a specific patient to be taken as a starting point for recruiting the patient for a study, the starting point used being for example a fundamental willingness of the patient to participate in the study before a suitable study is advantageously selected from the list.

Additionally, it is proposed that the ordered list is produced, in a computerized manner, in such a way that it is organized according to at least one criterion which is determined in a computerized manner, and/or according to at least one presettable criterion. Advantageously, the criterion is presettable by the patient and/or by a treating doctor and/or by other clinical personnel and/or by a party responsible for the study, for example. In particular, the list is organized according to the suitability parameter, preferably according to the number of matches between patient parameters and recruitment parameter and/or according to an overall match score and/or according to a probability of a match between the patient and the study, which is and/or was determined on the basis of a hierarchic matching of patient parameters and recruitment parameters, in particular. Alternatively or additionally, it is conceivable for the list to be organized according to a date, for example of a beginning of the study and/or of an inclusion of the patient and/or according to a period of time, for example a study period, in particular of fundamentally suitable studies, and/or a length of stay of the patient or the like. This advantageously allows a probability of a successful recruitment to be increased and/or a level of effort for a recruitment to be reduced.

In addition, it is proposed that at least one selection step involves a list of studies to be matched being produced, in particular on the basis of at least one piece of location information associated with the patient and/or with the study. Preferably, the matching step involves using merely studies that were added to the list of studies to be matched in the selection step. Preferably, the selection step takes place before the matching step. The location information can be for example a location of a healthcare institution at which the patient is currently staying and/or that the patient visits, preferably repeatedly, for outpatient treatment, for example, and/or a residence and/or a location of a family doctor and/or a location at which the study is performed, in particular a location of a healthcare institution at which the study is performed, and/or a whereabouts of a party responsible for the study or the like. Advantageously, studies are added to the list of studies to be matched that are and/or are supposed to be performed at the same healthcare institution at which the patient is currently staying and/or will, in particularly probably, stay in future. By way of example, it is conceivable that when the patient registers in the healthcare institution, which can in particular also cover at least information regarding a probable length of stay and/or regarding a room number of the patient and the like, available studies, the study data records of which are stored on the study data server, are searched for studies that have associated suitable location information, in particular in order to produce the list of studies to be matched. In particular if at least the study data server is in the form of a local server of the healthcare institution, it is conceivable for merely study data from studies performed at the healthcare institution to be stored and/or storable on said server. Alternatively, it is conceivable for study data pertaining to studies of different healthcare institutions to be stored and/or storable on the study data server.

Furthermore, it is conceivable for the list of studies to be matched to be produced on the basis of at least one date and/or a period of time and/or an appointment or the like. By way of example, the patient could be unsuitable for participation in a specific study, since he/she is already associated with another study at specific times and/or has a treatment appointment or the like, in which case the applicable study would preferably not need to be added to the list of studies to be matched. Similarly, it is conceivable that a patient's stay at the healthcare institution is probably, in particular considerably, shorter than a study period and, for that reason in particular, appears unsuitable for a specific study, which accordingly should advantageously not be added to the list of studies to be matched.

In an advantageous configuration of the invention, it is proposed that the selection step and/or the matching step and/or the association step are performed repeatedly. Preferably, the list of studies to be matched is worked through in order to produce the ordered list of suitable studies and preferably to associate it with the patient. Particularly preferably, the list of studies to be matched is repeatedly updated, in particular in real time, and/or repeatedly worked through, in particular in real time. Advantageously, patient parameters are matched with recruitment parameters in real time and/or suitability parameters are determined in real time. Preferably, the selection step and/or the matching step and/or the association step are performed repeatedly in automated fashion, for example daily or weekly and/or at specific, in particular regular and/or presettable, intervals of time, for example in order to check newly added studies for whether the patient is suitable for newly added studies. Moreover, the selection step and/or the matching step and/or the association step is advantageously performed afresh at least once after additional data, for example in the form of patient parameters and/or study parameters, have been input, in particular by the patient and/or by clinical personnel and/or by a party responsible for the study. This advantageously allows a high level of up-to-dateness to be attained. Moreover, it allows a high level of probability of a successful recruitment to be attained over a total period of a stay of a patient at a healthcare institution.

In a particularly advantageous configuration of the invention, it is proposed that at least one output step involves at least one piece of study information being output by means of at least one input and/or output unit. Preferably, the input and/or output unit is in the form of an input and output unit that is configured for an output of at least one piece of information for a user and that is configured for an input of at least one piece of information by a user, in particular the same user. Advantageously, study information for a study for which the patient is at least partially suitable is output. In particular, the input and/or output unit is configured to output and/or present study data for the studies on the ordered list of suitable studies, in particular in organized form. Advantageously, the input and/or output unit is configured to output, for output studies, a respective applicable suitability parameter or a suitability criterion determined on the basis of this suitability parameter, for example in the form of a number of points, a star rating, a bar filled to a greater or lesser extent according to suitability, a match score or the like. In addition, the input and/or output unit is advantageously configured to output the study information in different levels of detail, for example on the basis of an applicable selection and/or preselection by the patient and/or the clinical personnel. Further, it is conceivable for the input and/or output unit to be configured for pushing study information, for example in the event of a suitability parameter of the patient for a specific study increasing and/or in the event of a study having a high match score being newly added or the like. Preferably, the input and/or output unit is configured for arrangement in at least one healthcare institution in particular in at least one patient room and/or in at least one doctors' room and/or in at least one waiting room. Particularly preferably, the input and/or output unit is arranged and advantageously, in particular permanently, installed in at least one healthcare institution, in particular in at least one patient room and/or in at least one doctors' room and/or in at least one waiting room. The input and/or output unit can comprise a plurality of input and/or output devices, at least some of which may be of identical or different design. By way of example, the input and/or output unit can comprise different input and/or output devices for patient rooms, doctors' rooms and/or waiting rooms. This advantageously allows study data, in particular pertaining to suitable studies, to be provided in a simple and/or efficient manner.

Advantageously, the input and/or output unit is part of the patient recruitment system. Particularly preferably, the input and/or output unit is connectable and/or connected to the patient recruitment server. In particular, the input and/or output unit is configured for the input of at least one patient parameter and/or a participation intention and/or for the output of at least one piece of study information. The study information can comprise for example a textual and/or graphical and/or audible and/or audiovisual description of a specific study and/or an aim of the study and/or participation prerequisites for the study and/or contact information for a responsible person or the like. Preferably, the study information is suitable for allowing the patient to obtain information about a specific study. In particular, at least one input and/or output device, configured for use by a patient, of the input and/or output unit is configured for the input of the patient parameter. In particular in this case, the patient parameter can be for example patient-specific information suitable for adding to a patient data record but not yet stored on the patient data server. Alternatively or additionally, it is conceivable for the patient parameter to be input by means of the input and/or output unit by a clinical personnel, for example by a treating doctor. Preferably, at least information regarding an intention and/or willingness of the patient to participate in a particular study is inputtable as the participation intention by means of the input and/or output unit, in particular by the patient and/or by a clinical person associated with the patient. This advantageously allows information regarding potentially suitable studies to be provided in an efficient and/or comprehensive manner, in particular for a specific patient and/or for clinical personnel treating said patient.

Advantageous properties in respect of a user-friendliness and/or an effective transmission of information can be attained in particular if the input and/or output unit comprises at least one display, in particular a touch display. Preferably, the display is arranged in a patient room, in particular in the patient room of the patient. Alternatively or additionally, arrangement in a doctors' room and/or in a waiting room is conceivable. In particular, the input and/or output unit can have a plurality of displays by means of which information is inputtable and/or retrievable by patients and/or by clinical personnel. Particularly preferably, the display is at least part of an input and/or output device associated with the patient. Preferably, the display is installed in the patient room. Alternatively, it is conceivable for the display and/or the input and/or output device associated with the patient to be of removable design, for example in the style of a portable display and/or computer. Preferably, at least location information of the input and/or output device associated with the patient is stored on the patient data server, for example in the form of coordinates and/or in the form of a room number or floor information or the like.

Moreover, it is conceivable for the input and/or output unit to comprise at least one mobile apparatus. By way of example, the mobile apparatus may be associated with and/or belong to the patient and/or the clinical personnel, in particular the treating doctor. The mobile apparatus can be in particular in the form of a smartphone, a tablet PC, smart glasses, a smart watch, a smart band, a smart cloth, a smart ring, any other wearable or the like. Preferably, the mobile apparatus is configured for connection to at least the patient recruitment server. In particular, the mobile apparatus forms at least one input and/or output device of the input and/or output unit. This advantageously allows a high level of user convenience to be attained. Moreover, a high level of cost efficiency can be attained if for example mobile devices that are present anyway are integrated into the patient recruitment system.

In an advantageous configuration of the invention, it is proposed that the input and/or output unit is used to input at least one participation intention parameter of the patient and to store it on at least one server, in particular on the patient recruitment server and/or on the patient data server. The participation intention parameter can be a Boolean parameter that can assume the value “participation intended”, for example. It is conceivable for information regarding participation by the patient not being intended to be stored on the patient recruitment server in automated fashion and/or as a standard value, in particular for all studies on the ordered list of suitable studies, and for this information preferably to be erased for a specific study if an applicable participation intention parameter is input for this specific study. It is also conceivable for the participation intention parameter to contain a rating. By way of example, at least some studies on the ordered list of suitable studies can have a preference of the patient stored for them, said preference being organized in particular according to an interest by the patient in participating. In particular, the participation intention parameter can be input by the patient and/or by the clinical personnel, advantageously selectively. Moreover, it is conceivable for the input and/or output unit to be used to make available information regarding a participation intention and/or regarding a fundamental interest by the patient to a clinical personnel. By way of example, a treating doctor can be informed about a fundamental interest in a specific study by a patient and can preferably subsequently make contact with the patient regarding the study and/or can inform a party responsible for the study. This advantageously allows a probability of a successful recruitment to be increased.

Additionally, it is proposed that least one output parameter for the output of the study information is adapted, preferably in a computerized manner, on the basis of at least one patient feature, in particular at least one patient parameter. The patient feature can be for example an age and/or a sex and/or a level of education and/or a medical history and/or a personal preference and/or any suitable patient parameter. In particular, the patient recruitment system and/or the input and/or output unit is configured for context-sensitive presentation of information, in particular of the study information. Preferably, the output parameter is a presentation parameter, in particular of the display. By way of example, a font size and/or a display brightness and/or a language and/or a level of detail and/or formulation of content and/or of volume and/or an arrangement of partial information on an information area, for example on a display, or the like is adapted, in particular in automated fashion. Moreover, it is conceivable for technical terms, for example in study descriptions, to be replaced in automated fashion with patient-friendly language and/or with familiar terms, in particular on the basis of at least one patient parameter such as for example a preference of the patient. By way of example, the term “pneumonia” could optionally be replaced with the term “lung infection” or the like. Moreover, it is conceivable for at least one automated semantic analysis of a study description to be used to produce at least one simplified and/or more easily understandable and/or shortened study description. Furthermore, it is conceivable for the patient feature to be captured in automated fashion, for example by means of at least one query of at least one patient parameter from the patient data server and/or by means of at least one query of information stored on an external server, for example, or the like. It is also conceivable for the input and/or output unit to be configured for input of at least one target output parameter by the patient and/or by the treating clinical personnel, which target output parameter stipulates the output parameter. This advantageously allows information to be presented in a manner suited specifically to one patient and, in particular, allows the patient to be encouraged to participate in a study.

Moreover, it is proposed that at least one hierarchic rating list containing a plurality of rating criteria is produced in a computerized and/or in an automated fashion on the basis of a plurality of recruitment parameters for the study, on the basis of which the suitability parameter is determined. In particular, the rating criteria are determined from the recruitment parameters in a computerized fashion. Naturally, it is also conceivable for the rating list to be produced and/or presettable manually at least in part, for example by a party responsible for the study. Preferably, the rating list comprises a hierarchically structured logical comparison rule for comparing the recruitment parameters with the patient parameters. As described above, rating criteria in this case may be inclusion and/or exclusion criteria. Moreover, it is conceivable for rating criteria to be able to produce a plurality of different, in particular continuously distributed, values, for example probabilities and/or numbers of points and/or partial match scores or the like. Preferably, the rating step involves the rating list being used to determine a match score, in particular on the basis of a plurality of partial match scores, and/or a number of matches between recruitment parameters and patient parameters as suitability parameters. It is conceivable for the rating list and/or applicable recruitment parameters to be produced in at least partially automated fashion on the basis of a textual description of a study. This advantageously also allows complex prerequisites that need to be met for a suitability for a study to be determined and/or checked efficiently.

In an advantageous configuration of the invention, it is proposed that at least one respective weighting parameter is associated with the rating criteria, in particular in a computerized and/or in an automated fashion. It is conceivable for the weighting parameters to be produced in at least partially automated fashion, for example including in the form of starting values produced in automated fashion, which can be overwritten subsequently, in particular. By way of example, at least some of the weighting parameters may be adaptable by a party responsible for the study and/or by a customer and/or by a clinical personnel associated with an applicable study, or the like, in particular by means of the input and/or output unit. By way of example, a weighting parameter of a specific rating criterion can be changed in order to identify a larger or a smaller number of patients as potentially suitable and/or in order to make adjustments in the recruitment, for example to new insights and/or partial results from studies. Furthermore, it is conceivable for at least one weighting parameter to be determined in a computerized manner on the basis of at least one piece of study information, for example by means of at least one semantic analysis of a textual description and/or an analysis of a study aim or the like. This advantageously allows a level of quality of an identification of suitable patients to be improved.

Furthermore, it is proposed that at least one list of rating criteria requiring at least one input at least by a clinical personnel is produced. By way of example, this may be a list of rating criteria produced in automated fashion that is provided to the clinical personnel for checking and/or for clearance. Furthermore, it is conceivable for it to be a list of rating criteria that were not and/or cannot be added to the list of rating criteria in completely automated fashion and/or that were not and/or cannot be finished in completely automated fashion. Alternatively or additionally, it is conceivable for the list of rating criteria to be checked and/or given clearance and/or edited and/or added to and/or reorganized in particular by means of the input and/or output unit, by a party responsible for the study and/or by a clinical personnel. This advantageously allows a high level of flexibility to be attained for a formulation of rating criteria and/or rating strategies.

Advantageous properties for a provision of information for a patient interested in a study can be attained in particular if at least one piece of contact information pertaining to the study is provided for the patient. In particular, contact information is provided for studies in which the patient at least intends to participate. Further, contact information is advantageously provided for studies for which the patient and/or the clinical person has input, in particular by means of the input and/or output unit, at least one piece of information regarding an interest in the study. By way of example, the patient is provided with information pertaining to the studies on the ordered list of suitable studies, which the patient decides, for example for the purpose of closer consideration, in particular by means of at least one input of a selection by means of the input and/or output unit. It is conceivable for the input and/or output unit to be configured for additionally making contact with a contact for the study, for example in the form of a call and in the form of a notification or the like.

A high level of reliability for determining a suitability and/or for recruiting suitable patients can be attained in particular if at least one input step involves at least one input and/or output unit being used to query and/or input at least one further patient parameter. In particular, the further patient parameter is requested by the patient and/or via the clinical personnel. Preferably, a type of the further patient parameter is determined in automated fashion, for example in the event of a partial match between the patient data record and the study data record, in particular for the complete or at least more complete identification of a suitability parameter. The further patient parameter can be any patient parameter that is in particular not yet stored on the patient data server and/or is advantageously stored on the patient data server after being input.

Furthermore, it is proposed that the input and/or output unit, in particular an input and/or output device associated with the clinical personnel, is used to make available at least one piece of suitability information for the patient to a clinical personnel associated with the patient. In particular, the input and/or output unit is configured to make the ordered list of studies and also suitability criteria and/or suitability parameters associated with the studies available, in particular to output and/or display them. Preferably, the input and/or output unit is configured to process a selection of different patients by the clinical personnel and preferably to make applicable suitability information, associated with a respective selected patient, and also particularly advantageously associated study information available, for example on a visit and/or when looking over an occupancy or the like. Moreover, it is conceivable for the input and/or output unit to be configured to provide and/or output at least one piece of study information to the patient on the basis of at least one preselection by a clinical personnel, the preselection preferably being able to be input by the clinical personnel following output of the suitability information of the patient for the clinical personnel. This advantageously allows a high level of cost efficiency and/or a time saving to be attained, in particular because clinical personnel can encourage potentially suitable patients specifically to participate in clinical studies.

In an advantageous configuration of the invention, it is proposed that at least one patient parameter is captured in automated fashion and is processed in a computerized manner by at least one sensor unit, in particular a sensor unit of the input and/or output unit. Preferably, the patient recruitment system has the sensor unit. The sensor unit may be installed, in particular permanently, in a patient room and/or on a patient bed, for example. It is also conceivable for the sensor unit to be in the form of a mobile sensor unit, which may be able to be taken in particular to different positions and/or to different patient bed and/or to different patient rooms. Moreover, it is conceivable for the sensor unit to be able to be fitted to a body of the patient, for example in the form of a band and/or a belt and/or a head covering and/or a contact point or the like. The sensor unit can also comprise at least part of an article of clothing of the patient. It is also conceivable for the sensor unit to be at least part of the mobile apparatus. The sensor unit can comprise a body temperature sensor and/or an image capture unit and/or a weight sensor and/or a body parameter sensor and/or a fingerprint scanner or the like. By way of example, the sensor unit can comprise a camera and/or a motion sensor and/or another sensor, for example of a smart phone and/or smart glasses and/or a wearable or else of an input and/or output device arranged, in particular permanently installed, on the patient bed and/or in the patient room, or the like. Preferably, the sensor unit is configured to transmit at least one captured parameter and/or at least one piece of status information and/or any data to at least one server, in particular to the patient data server and/or to the study data server and/or to the patient recruitment server. The sensor unit may be configured for example for determining a body weight and/or a body temperature and/or a fingerprint and/or a sleeping behavior and/or a tiredness and/or any other patient parameter. Moreover, it is conceivable for the sensor unit to be configured for image capture. In particular in this case, it is conceivable for the patient recruitment server and/or the input and/or output unit and/or the sensor unit to be configured for image recognition, on the basis of which in particular at least one patient parameter is determinable. By way of example, the sensor unit can be used to capture at least one image, for example of the patient and/or of a medicament package and/or of a wound and/or of a body part and/or of a dressing of the patient or the like, on the basis of which at least one patient parameter is determinable. In particular, it is conceivable for the method to have a method step for determining a piece of medicament use information for the patient by virtue of the patient being asked for example to scan in and/or photograph prescriptions and/or medicament packages and/or tablets or the like, and/or which method step involves the patient taking appropriate pictures and/or performing scans and/or which method step advantageously involves accordingly captured pictures and/or scans being analyzed in a computerized fashion for the purpose of capturing the applicable patient parameters in a computerized manner and/or storing them on the patient data server in an automated fashion.

A high level of user convenience and/or effective recruitment on the basis of a patient-specific representation can be attained in particular if at least one output parameter for an output of at least one piece of study information is adapted on the basis of the patient parameter captured by means of the sensor unit. By way of example, it is conceivable for the output parameter to be adapted on the basis of an identified age and/or an identified deterioration, for example of a vision of the patient, and/or on the basis of medicaments currently prescribed for the patient that are identified by means of the sensor unit, and/or an identified tiredness of the patient or the like. Moreover, it is conceivable for the sensor unit to be configured for detecting at least one uniqueness feature of a patient, such as for example a fingerprint, a face, an eye or else for example an RFID chip or the like that the patient carries on his body and/or that is integrated in an article of clothing of the patient, in particular one provided by the healthcare institution. It is also conceivable for a feature that is not necessarily unique but that is associated with the patient, such as for example a body weight, a sex, an estimated age, an eye color and/or the like, in particular also in combination, to be stored and/or to be compared with current detections by the sensor unit in order to identify a patient. By way of example, it is possible to identify whether a different patient than one envisaged in accordance with an occupancy plan is in a specific patient bed and/or uses a specific input and/or output device and/or wishes to query specific study information intended for the correct patient, or the like. In particular in this case, it is conceivable for at least one display and/or another output means of the input and/or output unit to be at least temporarily deactivated and/or disabled if a different patient, other than the patient, and/or no patient is detected. Advantageously, this allows the display of sensitive and/or personal data to unauthorized people to be avoided.

Advantageous properties in respect of a convenience of use and/or an efficient patient recruitment can be attained in particular by means of an input and/or output unit that is configured for use in a patient recruitment system according to the invention.

The invention relates moreover to the use of the method according to the invention and/or of the patient recruitment system according to the invention for recruiting at least one patient for at least one study, in particular for a clinical study, advantageously for a phase III clinical study, the aim of which is testing a therapeutic effect of an active substance and in particular the approval thereof, for example. Naturally, use for recruiting patients for any other, in particular clinical study is also conceivable.

The method according to the invention and the patient recruitment system according to the invention are not meant to be restricted to the applications and embodiments described above. In particular, the method according to the invention and the patient recruitment system according to the invention can comply with a mode of operation described herein by having a different number of individual elements and/or components and/or units and/or method steps than that cited herein. In particular, the method according to the invention can contain specific method steps in which at least one of the features of the patient recruitment system according to the invention that are described above and an associated functionality are implemented in part in each case. Moreover, in the case of the value ranges indicated in this disclosure, values within the cited limits are also meant to be deemed to have been disclosed and to be useable as desired. Unless indicated otherwise, it is furthermore conceivable for all of the method steps and/or processes described to be executed, at least in part and advantageously in full, in a computerized and/or in an automated fashion and/or for the patient recruitment system to be configured for at least partial and advantageously fully computerized and/or automated execution thereof and/or to comprise an applicable process and/or to be configured for the execution thereof. Moreover, “in a computerized fashion” within the context of this disclosure can in particular also cover “in automated fashion”, wherein computerized, non-automated processes are of course also conceivable.

### DRAWINGS

Further advantages arise from the description of the drawings that follows. The drawings show an exemplary embodiment of the invention. The drawings, the description and the claims contain numerous features in combination. A person skilled in the art will advantageously also consider the features individually and combine them to produce useful further combinations.

In the drawings:

FIG. 1 shows a healthcare institution having a patient recruitment system in a schematic depiction,

FIG. 2 shows a schematic flowchart for a method for recruiting patients,

FIG. 3 shows a hierarchic rating list in a schematic depiction, and

FIG. 4 shows an input and output device of an input and/or output unit of the patient recruitment system in a schematic depiction.

### DESCRIPTION OF THE EXEMPLARY EMBODIMENTS

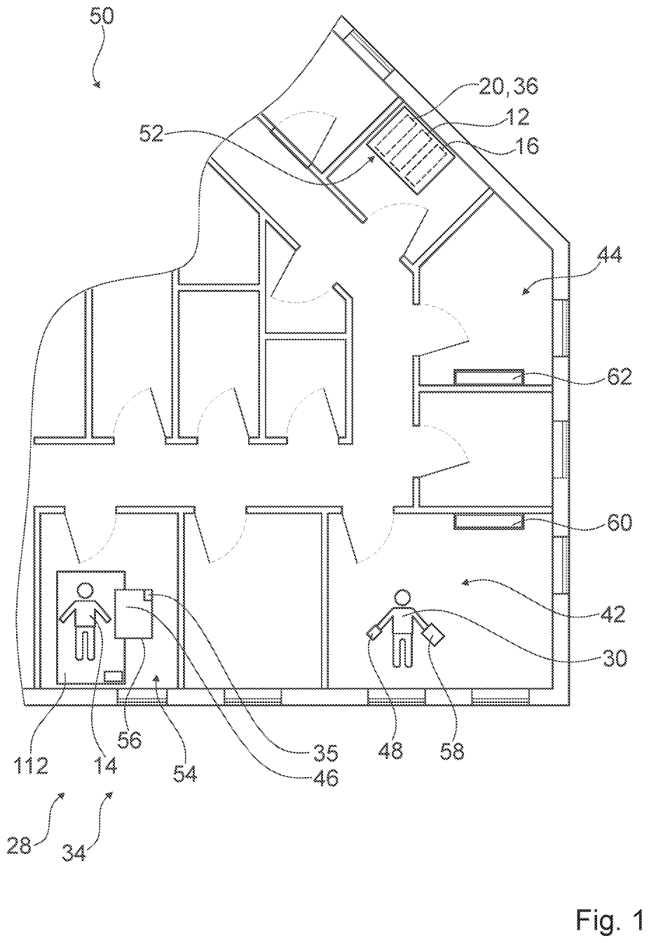

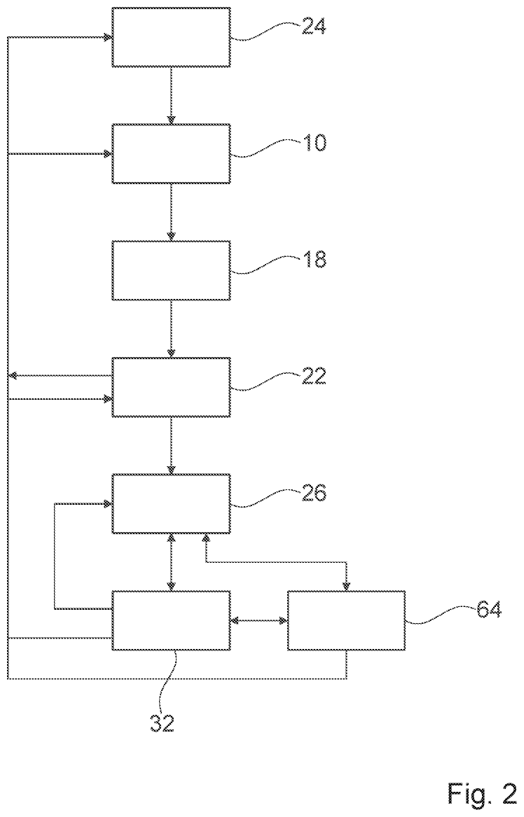

FIG. 1 shows a healthcare institution 50 having a patient recruitment system 34 in a schematic depiction. The healthcare institution 50 can be a doctor's practice, a clinic or preferably a hospital or the like, for example. The patient recruitment system 34 in the present case is configured to perform a method for recruiting patients for a clinical study. A schematic flowchart for the method for recruiting patients is depicted in FIG. 2. The patient recruitment system 34 and/or the depicted method for recruiting patients can be used to recruit at least one patient 14 for at least one study, in particular for a clinical study, advantageously for a phase III clinical study, the aim of which is testing a therapeutic effect of an active substance and in particular the approval thereof, for example.

The patient recruitment system 34 has at least one patient recruitment server 36. In the present case, the patient recruitment system 34 further has at least one patient data server 12 and at least one study data server 16. The patient data server 12, the study data server 16 and the patient recruitment server 36 in the present case are each part of a common dedicated server 52 incorporated into an intranet of the healthcare institution 50. In particular, the patient data server 12, the study data server 16 and the patient recruitment server 36 are connected to one another and/or arranged in a common network. However, it is naturally conceivable for at least some of the patient data server 12, the study data server 16 and/or the patient recruitment server 36 to be separate from one another and/or for at least some of them to be designed to be outside the healthcare institution 50 and/or its intranet and/or in the form of a cloud or the like.

In the case depicted, a patient 14 is staying in a patient room 54 of the healthcare institution 50. The patient 14 can be or may have been treated as an inpatient, for example. The patient 14 has an associated clinical personnel 30, for example a treating doctor. The clinical personnel 30 can be formed in particular by a plurality of people, for example doctors and/or nurses and/or physiotherapists or the like, for example in shifts or the like.

In the present case, a plurality of clinical studies exist that are performed directly at the healthcare institution 50 at present and/or in future, for example. In the present case, study data records pertaining to these studies are stored on the study data server 16. Preferably, only study data pertaining to the studies of the healthcare institution 50 are stored on the study data server 16. However, it is likewise conceivable for, alternatively or additionally, study data pertaining to studies performed in other places and/or healthcare institutions at present and/or in future to be stored. Study data and in particular study data records can be or may have been produced and/or stored on the study data server 16 by parties responsible for the studies, for example. At least some study data can be produced in automated fashion in this case, for example on the basis of inputs into an applicable input mask. In particular, incomplete study data records may also be storable and/or stored on the study data server 16.

Moreover, in the present case, a plurality of patient data records exist that are stored on the patient data server 12. Patient data records may be associated both with former and with current and/or future patients. Preferably, a specific patient data record contains at least parts of a treatment past of the respective patient 14, the sex of said patient, the age of said patient, the medication of said patient and/or any other patient data. Patient data can be based, for example on inputs and/or details from patients, from clinical personnel, from a health insurance company of an applicable patient and the like and/or can be and/or may have been produced in automated fashion at least in some cases. In particular, incomplete patient data records may also be stored and/or storable on the patient data server 12.

Moreover, a plurality of recruitment data records exist in the present case, these being stored on the patient recruitment server 36. A recruitment data record in this instance advantageously contains at least one recruitment parameter. In particular, a recruitment data record for a specific study contains details regarding specific features that a patient 14 should and/or needs to have in order to be suitable for participation in the specific study. By way of example, an age of test subjects, the medication of said test subjects, the sex of said test subjects, the medical history of said test subjects, the weight of said test subjects and the like can form applicable recruitment parameters. Preferably, recruitment parameters are able to be input by a party responsible for the study. It is also conceivable for recruitment parameters to be produced in automated fashion, in particular on the basis of at least one input. Any manner of formulation of such parameters is conceivable for this, for example as a logic function, as an input in a suitable input mask or the like. Moreover, a computerized formulation of recruitment parameters, for example on the basis of at least one semantic analysis of at least one study description and/or of a requirement profile, is conceivable. Fundamentally, incomplete recruitment data records may also be storable and/or stored on a patient recruitment server 36.

In the present case, patient data are fundamentally interchanged between the patient data server 12, the study data server 16 and the patient recruitment server 36 in anonymized and/or pseudonymized form.

The patient recruitment server 36 is configured to execute at least one matching process to match at least one patient parameter of at least one patient 14 with at least one recruitment parameter of at least one study. Further, the patient recruitment server 36 is configured to execute at least one rating process to generate at least one suitability parameter of suitability of the patient 14 for the study on the basis of the patient parameter and the recruitment parameter. Moreover, the patient recruitment server 36 is configured to execute at least one association process to produce at least one association between the study and the patient 14 on the basis of the suitability parameter.

As mentioned above, the patient recruitment system 34 is used to perform a method for recruiting patients with clinical studies, the sequence of which method is depicted schematically in FIG. 2. The depiction of method steps and/or sequences of the method is purely exemplary in this case and in particular not to be understood as final. Moreover, any method steps can be repeated and/or carried out in an order other than that depicted.

The method for recruiting patients comprises at least one matching step 10 in which at least one patient parameter, stored on the patient data server 12, for at least one patient 14 is matched, in a computerized manner, with at least one recruitment parameter, stored on the study data server 16, for at least one study. In particular, the matching step 10 is performed by means of the matching process. Moreover, the method for recruiting patients comprises at least one rating step 18 in which at least one suitability parameter of a suitability of the patient 14 for the study is determined, in a computerized manner, on the basis of the patient parameter and the recruitment parameter and stored on at least one server 20, in particular on the patient recruitment server 36. In particular, the rating step 18 is performed by means of the rating process. Additionally, the method for recruiting patients comprises at least one association step 22 in which at least one association between the patient 14 and the study is made on the basis of the suitability parameter, and at least one piece of association information is stored on at least one server, in particular on the patient recruitment server 36.

The association step 22 involves the study being associated with the patient 14 on the basis of the suitability parameter. In particular, the patient 14 serves as a starting point for searching for suitable studies, which are associated with the said patient if he/she is suitable.

The association step 22 involves a plurality of suitable studies being associated with the patient 14. In particular, a respective number of suitable studies associated with a plurality of or all patients 14 that are in the healthcare institution 50 are stored on the patient recruitment server 36 for said patients. Preferably, the patient recruitment server 36 stores for each patient 14 a piece of information indicating those studies, which are performed at the healthcare institution 50, in particular, for which the patient 14 appears fundamentally suitable or at least partially suitable and/or at least not unsuitable.

In the present case, an ordered list of suitable studies, which is stored on at least one server 20, in particular on the patient recruitment server 36, is associated with the patient 14 in the association step 22. Preferably, a respective individual ordered list of suitable studies is stored on the patient recruitment server 36 for a plurality of or all patients 14 that are in the healthcare institution 50. It is conceivable for an applicable ordered list to be produced in automated fashion for each patient 14 that is currently in the healthcare institution 50, for example the first time that he/she registers and/or is admitted and/or is received. It is also conceivable for an ordered list of suitable studies not to be produced until there is at least clearance, this being given by the clinical personnel 30 and/or by the patient 14 himself, for example. By way of example, it would be conceivable for the patient 14, on admission, to have to agree that his/her suitability for studies can fundamentally be checked by the patient recruitment server 36.

The ordered list is produced in a computerized manner, and is organized according to at least one criterion that is determined in a computerized manner, and/or according to at least one presettable criterion, as in particular also indicated above. By way of example, the ordered list may be organized according to a match score for the patient 14 for the studies on the list, with a best-suited list advantageously forming a first or a last entry on the list. The list can if necessary also be organized according to a date, for example a starting date for the applicable studies, according to a location at which the studies are performed, according to a study period and/or the like. In particular, it is conceivable for the list to be organized on the basis of at least one input by the patient 14 and/or by the clinical personnel 30. In particular, the patient 14 can have an appropriately organized list produced by inputting a specific preference.

In at least one selection step 24, a list of studies to be matched is produced. In particular, the list of studies to be matched is produced on the basis of at least one piece of location information associated with the patient 14 and/or with the study. Further, it is conceivable for the list of studies to be matched to be produced on the basis of a length of stay and/or a date of stay of the patient 14 and/or a starting date and/or a study period of the study or the like. By way of example, it is conceivable for merely studies that are begun and/or performed at the healthcare institution 50 while the patient 14 is staying at the healthcare institution 50 to be put onto the list of studies to be matched. Alternatively, it is conceivable for studies performed at other healthcare institutions, for example for the mediation of the patient, to be put onto the list of the studies to be matched.

The patient recruitment system 34 has an input and/or output unit 28, connectable to the patient recruitment server 36, that is configured for the input of at least one patient parameter. The input and/or output unit 28 is depicted schematically in FIG. 1. In the present case, the input and/or output unit 28 is an input and output unit for inputting information, which are can be stored on the patient recruitment server 36, for example, and for outputting information, for example study data and/or patient data and/or suitability parameters, in particular for the patient 14 or for the clinical personnel 30. Furthermore, the input and/or output unit 28 is configured for the input of at least one participation intention, in particular an intention of the patient 14 to participate in the study. Moreover, the input and/or output unit 28 in the present case is configured for the output of at least one piece of study information.

The input and/or output unit 28 is configured for arrangement in the healthcare institution 50, in particular, the input and/or output unit 28 is configured for arrangement in at least one patient room 54 and/or in at least one doctors' room 42 and/or in at least one waiting room 44.

In the present case, the input and/or output 28 comprises a first input and output device 56. The first input and output device 56 is arranged in the patient room 54. The first input and output device 56 may be secured and/or installed, in particularly permanently, on a patient bed 112 and/or on a wall and/or ceiling of the patient room 54, for example. It is also conceivable for the input and output device 56 to be in the form of a portable input and output device, for example in the style of a portable computer, a tablet PC, headphones, smart glasses or the like. The patient 14 can obtain information about studies, in particular studies running at the healthcare institution 50, in particular studies on his/her associated list of suitable studies, by means of a first input and output device 56. The input and/or output unit 28 comprises at least one display 46 arranged in the patient room 54. In the present case, the input and output device 56 has the display 46. The display 46 may be in the form of a touch display. In particular, the display 46 is configured for input and/or output of information, in particular of study data and/or suitability data and/or patient data.

Further, the input and/or output unit 28 in the present case comprises a second input and output unit 58 associated with the clinical personnel 30. The second input and output unit 58 can differ in terms of functionality and/or in terms of a configuration and/or in terms of actions performable therewith or else may be identical thereto. In the present case, the second input and output device 58 is designed for example in the style of a tablet PC or an electronic paper or the like and can advantageously be used at different locations by the clinical personnel 30, for example on a visit. In this instance, it is in particular conceivable for the second input and output device 58 to have at least one sensor configured for automated detection of a room number and/or for identification of patients 14. In particular, it is conceivable for the clinical personnel 30 to be provided with information on the basis of a detection by the sensor, for example the list of suitable studies associated with the patient 14 when the patient room 54 of the patient 14 is entered.

Further, the input and/or output unit 28 in the present case comprises a third input and output device 60. The third input and output device 60 may be arranged in a doctors' room 42 at the healthcare institution 50, for example. In the present case, the third input and output device 60 is in the form of a permanently installed touch display by means of which study data and/or patient data and/or ordered lists of suitable studies for different patients 14 can be retrieved. In particular, it is conceivable for the third input and output device 60 to be configured to output at least one notification, in particular unprompted, for example if a patient 14 having a high match score with the specific study has been admitted to the healthcare institution 50.

Moreover, the input and/or output unit 28 in the present case comprises a fourth input and output device 62. The fourth input and output device 62 may be arranged in a waiting room 44, for example. The fourth input and output device 62 may be configured to display study data to a patient 14, for example during a waiting time, in particular when the patient 14 is registered, which allows his patient data record to be retrieved from the patient data server 12.

Moreover, it is conceivable for the input and/or output unit 28 to comprise at least one mobile apparatus 48. In the present case, the input and/or output unit 28 comprises at least one mobile apparatus 48 of the clinical personnel 30, for example a work smartphone. The mobile apparatus 48 advantageously forms at least one input and output device of the input and/or output unit 28. The mobile apparatus 48 may be configured for example to execute at least one program, in particular an app, allowing incorporation into the patient recruitment system 34. By way of example, the mobile apparatus 48 may also be in the form of a tablet PC, a smartwatch, a smart cloth, a smartband, any other wearable, a computer or the like. Moreover, it is conceivable for the mobile apparatus 48 to be a mobile apparatus of the patient 14.

Reference is once again made to FIG. 2 below. At least one output step 26 involves at least one input and/or output unit 28 being used to output at least one piece of study information. The study information is displayed to the patient 14. Alternatively or additionally, it is conceivable for the study information to be displayed to the clinical personnel 30. Preferably, the study information is generated on the basis of at least one study data record stored on the study data server 16, for example by using a study description and/or contact information and/or location information and/or a study period or the like. In this case, it is conceivable for different study information to be output to the patient 14 than to the clinical personnel 30. In particular, the study information is conditioned for the patient 14 in patient-friendly fashion, for example in regard to language and/or a level of detail and/or use of further background information that the patient 14 needs, for example, but that is common general knowledge in the art for clinical personnel 30, for example.

Moreover, at least one method step 64 involves the input and/or the output unit 28 being used to input at least one participation intention parameter of the patient 14 and to store it on at least one server, in particular on the patient recruitment server 36. In particular, the participation intention parameter is a participation intention parameter associated with the study. In this case, the participation intention parameter can assume the values “participation intended” or “participation not intended”, for example. Moreover, it is conceivable for the participation intention parameter to indicate that the patient 14 is fundamentally interested in the study, but for example wants additional information, in particular provided by a party responsible for the study and/or by clinical personnel 30, and/or would like to be reminded about the study again, for example in order to be given time to consider. It is conceivable for the ordered list of suitable studies, to which the participation intention information has been added, to be displayed to the patient 14 following input of the participation intention parameter, so that the patient 14 can obtain from a display the information regarding which studies he intends to participate in.

Further, at least one output parameter for the output of the study information, in particular in the output step 26, is adapted on the basis of at least one patient feature, in particular at least one patient parameter. By way of example, it is conceivable for the patient 14 to be able to adapt a type and/or scope of depicted information by means of inputs that he/she makes in particular using his/her associated first input and output device 56. By way of example, it is conceivable for the ordered list of suitable studies to be displayed to the patient 14 first and for said patient subsequently to be able to retrieve more detailed study information pertaining to individual studies, for example by tapping and/or clicking or selecting. Further, it is conceivable for different study information, for example for different target groups, to be stored for a specific study. It is then conceivable for a type and/or extent of a depiction of study information and/or a wording and/or additional external links or the like to be adapted, in particular in automated fashion, on the basis of one or more patient parameters, preferably independently of a patient input, for example on the basis of said patient's mother tongue and/or sex and/or age and/or any visual impairment or the like.

Additionally, at least one piece of contact information pertaining to the study can be provided for the patient 14. By way of example, a name and/or telephone number and/or location of a party responsible for the study can be displayed. It is furthermore conceivable for the input and/or output unit 28 to be configured to provide at least one communication channel, for example a call line and/or a video call line and/or a text message channel or the like that is preferably intended for the patient 14 to make contact with a party responsible for the study.

In particular the rating step 18 involves at least one hierarchic rating list with a plurality of rating criteria being produced in a computerized fashion on the basis of a plurality of recruitment parameters for the study, on the basis of which the suitability parameter is determined. The rating list may also have been produced, in particular as a whole, beforehand, in particular in automated fashion, and advantageously stored on the patient recruitment server 36 and/or on the study data server 16. In this case, it is conceivable for the rating list to be produced in automated fashion, at least in part, by using at least one semantic analysis of at least one textual description, in particular of a study description. It is also conceivable for the input and/or output unit 28 to be configured for input and/or support, in particular for a party responsible for the study, when producing the rating list.

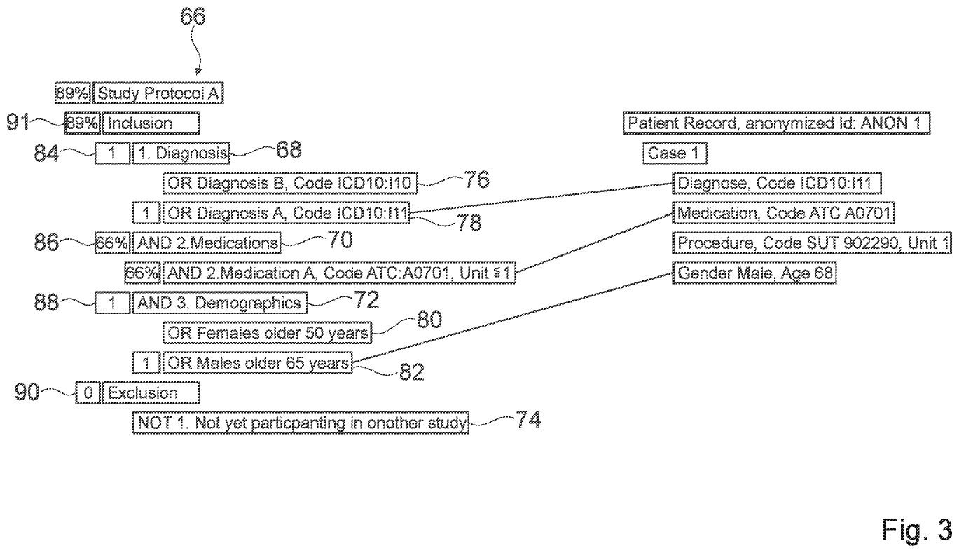

FIG. 3 shows a schematic depiction of an exemplary rating list 66 for an exemplary study. The exemplary rating list 66 is based on a specific study protocol.

The exemplary rating list 66 comprises a plurality of rating criteria 68, 70, 72 that can potentially lead to inclusion of a patient 14. In the present case, a first rating criterion 68 is a diagnosis criterion. According to study protocol, one of two possible diagnosis criteria 76, 78 should be met for a patient 14 who could potentially be suitable for the study, that is to say that one of two possible diagnoses should be available. The patient 14 complies with the first diagnosis, for example, in the present case. A partial match score 84 of 1 is therefore associated with the first rating criterion.

A second rating criterion 70 is a medication in the present case. The patient 14 meets this criterion in part, for example, in the present case, since he takes a similar medicament to that required in accordance with the second rating criterion 70. A partial match score 86 of 0.66, for example, is therefore associated with the second rating criterion 70.

A third rating criterion 72 is a demographic criterion, for example, in the present case. According to a study protocol, a patient 14 who could potentially be suitable for the study should meet one of two possible demographic criteria 80, 82. By way of example, a patient 14 should be female and older than 50 or male and older than 65. In the present case, the patient is male and 68 years old, for example. A partial match score 88 of 1 is therefore associated with the third rating criterion 72.

Moreover, the rating list 66 in the present case comprises a fourth rating criterion 74, which can potentially lead to exclusion of a patient 14. The fourth rating criterion 74 in the present case is an exclusion criterion. The fourth rating criterion 74 in the present case leads to exclusion of a patient 14, for example, if he/she is already participating in another study. In the present case, the patient 14 is not currently participating in another study, for example. A value 90 of 0 is therefore associated with the fourth rating criterion 74. It is conceivable for meeting of an exclusion criterion to lead to exclusion of a patient 14 independently of a rating of other rating criteria.

In the case of the exemplary rating list 66, a respective weighting parameter is associated with the rating criteria 68, 70, 72, 74. For each of the first, second and third rating criteria 68, 70, 72, the weighting factor is 1 in the present case. For this reason in particular, the overall match score is formed as an arithmetic mean. A weighted average could be formed for the different weighting parameters, for example. It is also conceivable for the weighting parameters to specify a hierarchic rating, so that for example specific partial match scores are ignored for the overall rating if other partial match scores are in specific ranges, for example are below and/or above specific limit values. Further, in the present case, a weighting parameter is associated with the fourth rating criterion 74 such that an overall match score of 0 is enforced if the fourth rating criterion 74 is met, that it to say the patient 14 is already participating in another study, for example. Weighting parameters can accordingly contain, by way of example, in particular discrete and/or constant functions and/or calculation rules and/or numerical values and/or categorizations and the like.

In the present case, an overall match score 91 of the patient 14 is determined for the exemplary study, for example as an arithmetic mean of partial match scores, if an exclusion criterion is not met. In the present case, the overall match score for the patient 14 is accordingly determined to be 0.89. The overall match score 91 and the applicable association between the patient 14 and the exemplary study are stored on the patient recruitment server 36.

It goes without saying that any other rating lists are conceivable that comprise a different number of criteria and/or hierarchy levels, etc. Moreover, instead of an arithmetic mean, it is conceivable for rating criteria to be weighted at different levels.

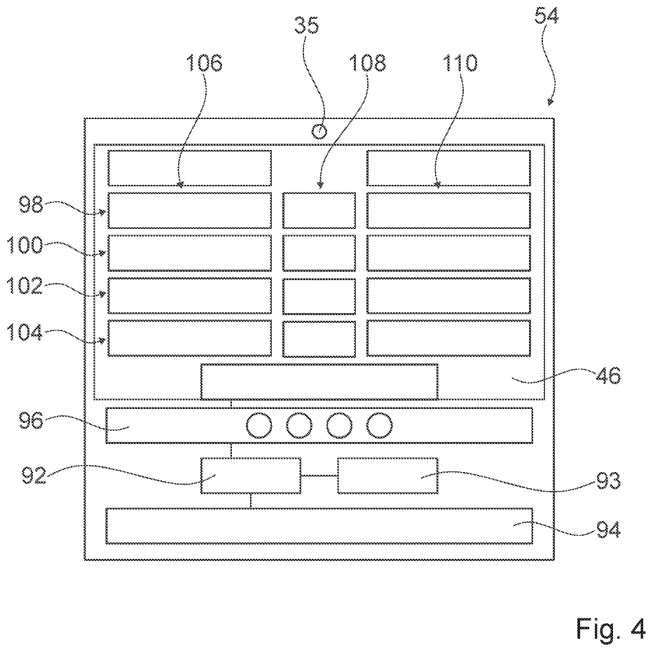

In particular analogously to the case of the exemplary study, the list of suitable studies and associated suitability parameters are determined and stored on the patient recruitment server 36. As mentioned above, this list can be made available to the patient 14 by means of the input and/or output unit 28, in particular by means of the first input and output device 56 associated with the patient 14. FIG. 4 shows a schematic depiction of the first input and output device 56. The input and output device 56 has the display 46. Moreover, the input and output device 56 has a computing unit 92 and an internal memory 93. Additionally, the input and output device 56 has an interface 94 for connection to a network, in particular for connection to the patient recruitment server 36. Further, the input and output device 56 has an input unit 96 that comprises a plurality of buttons, for example. It is also conceivable for the input unit 96 to comprise a mouse and/or a keyboard. Moreover, it is conceivable for the input unit 96 to be formed at least in part by the display 46, for example if the latter is in the form of a touch display.

In at least one operating state of the input and output device 56, information pertaining to at least some of the studies on the list of suitable studies, for example pertaining to the studies having the highest match scores, is displayed to the patient 14. In the depicted case, which is naturally to be understood purely by way of example, a study title (column 106), a match score (column 108) and contact information (column 110) are presented for each of four exemplary studies 98, 100, 102, 104, for example. By clicking or tapping on an applicable line of a presented list, for example, the patient 14 can retrieve further study information and/or input a participation intention and/or request further information, for example from the clinical personnel 30 and/or from a party responsible for the study. It goes without saying that any other presentations and/or types of information provision are conceivable.

Reference is once again made below to FIGS. 1 and 2. In the present case, for example in the matching step 10 and/or in the rating step 18 and/or at another suitable time, at least one list of rating criteria requiring at least one input at least by a clinical personnel 30 is produced. This list of rating criteria comprises in particular rating criteria that cannot be evaluated in automated fashion, for example on account of absence of appropriate patient parameters and/or on account of a need for input of a medical assessment or the like. Preferably, the clinical personnel 30 is asked, by means of the second input and output device 58, to input appropriate information. Advantageously, applicable inputs by the clinical personnel 30 are used to process, in a computerized manner, at least the affected rating criteria and/or are stored on the patient data server 12 and/or the study data server 16 and/or the patient recruitment server 36.

Further, at least one input step 32 involves the input and/or output unit 28 being used to request and/or input at least one further patient parameter. This can be a patient parameter that is needed in accordance with the described list of rating criteria. Advantageously, the further patient parameter is requested and/or input by the patient 14. By way of example, the patient 14 can be asked to complete his patient data record. In particular, the input step involves personal data and/or health data and/or preferences of the patient 14 or the like being input by the patient 14 and advantageously used to add to a patient data record associated with the patient 14.

Additionally, for example in the output step 26, the input and/or output unit 28 is used to make at least one piece of suitability information for the patient 14 available to the clinical personnel 30 associated with the patient 14. By way of example, the suitability information can be output by means of the second input and output device 58 and/or by means of the third input and output device 60. By way of example, the list of suitable studies can be displayed to the clinical personnel 30. In particular, the clinical personnel 30 is rendered able to make the patient 14 aware of his/her suitability of a specific study if need be. Fundamentally, it is conceivable for the clinical personnel 30 to be able to request study data and/or input information by means of the input and/or output unit 28 similarly to the patient 14.

In the present case, at least one patient parameter is moreover captured in automated fashion by means of at least one sensor unit 35 and processed in a computerized manner, for example, in the input step 32. In this instance, as mentioned above, any sensors are conceivable, these being fitted in the patient room 54 and/or on a patient bed 112, for example. In particular, they may be sensors present anyway on medical monitoring devices that can be incorporated into a network of the patient data server 12, for example. In the present case, the first input and output device 56 associated with the patient 14 has the sensor unit 35. The sensor unit 35 in the present case comprises a digital video camera. The sensor unit 35 can be used to take pictures and videos that can subsequently be used for an image analysis, for example. The sensor unit 35 can be used to take pictures of medicament packages of the patient 14, for example. An image analysis of these pictures is then used to produce a list of medicaments for the patient 14 that is displayed to him/her for the purpose of correctness checking and/or completion, for example. By way of example, after a specific medicament is identified, it is possible to ask since when and/or how often the patient 14 takes said medicament.

Moreover, at least one output parameter for an output of at least one piece of study information can be adapted on the basis of the patient parameter captured by means of the sensor unit 35. In the present case, for example the determined medication of the patient 14 is used to determine said patient's familiarity with specific symptoms in automated fashion. Further, an estimated familiarity of the patient 14 with specific symptoms is taken as a basis for adapting a level of detail of study descriptions, for example in order to provide the patient 14 with additional information pertaining to studies that could be of interest to him/her on the basis of his/her personal medical history and encourage him/her to participate. As mentioned above, any other adaptations of output parameter are conceivable. As such, by way of example, a sex and/or an age and/or a behavior parameter and/or an attentiveness of the patient 14 or the like can be determined on the basis of at least one sensor detection and an output parameter can be adapted accordingly.

1. A method for recruiting patients for a study, in particular for at least one clinical study, wherein at least one matching step involves at least one patient parameter of at least one patient, stored on a patient data server, being matched, in a computerized manner, with at least one recruitment parameter of at least one study, stored on a study data server, and wherein at least one rating step involves at least one suitability parameter of a suitability of the patient for the study being determined, in a computerized manner, on the basis of the patient parameter and the recruitment parameter and stored on at least one server, in particular a patient recruitment server, wherein in at least one association step at least an association between the patient and the study is made on the basis of the suitability parameter, and at least one piece of association information is stored on at least one server, in particular the patient recruitment server.

2. The method as claimed in claim 1, wherein the association step involves the study being associated with the patient on the basis of the suitability parameter.

3. The method as claimed in claim 2, wherein the association step involves a plurality of suitable studies being associated with the patient.

4. The method as claimed in claim 3, wherein the association step involves an ordered list of suitable studies being associated with the patient, said list being stored on at least one server, in particular the patient recruitment server.

5. The method as claimed in claim 4, wherein the ordered list is produced, in a computerized manner, in such a way that it is organized according to at least one criterion which is determined in a computerized manner, and/or according to at least one pre-settable criterion.

6. The method as claimed in claim 1, wherein at least one selection step involves a list of studies to be matched being produced, in particular on the basis of at least one piece of location information associated with the patient and/or with the study.

7. The method as claimed in claim 6, wherein the selection step is performed repeatedly.

8. The method as claimed in claim 1, wherein at least one output step involves at least one piece of study information being output by means of at least one input and/or output unit.

9. The method as claimed in claim 8, wherein the input and/or output unit is used to input at least one participation intention parameter of the patient and to store it on at least one server, in particular the patient recruitment server.

10. The method as claimed in claim 8, wherein at least one output parameter for the output of the study information is adapted on the basis of at least one patient feature, in particular at least one patient parameter.

11. The method as claimed in claim 1, wherein at least one hierarchic rating list containing a plurality of rating criteria is produced in a computerized manner on the basis of a plurality of recruitment parameters of the study, on the basis of which the suitability parameter is determined.

12. The method as claimed in claim 11, wherein at least one weighting parameter is associated with each of the rating criteria.

13. The method as claimed in claim 11, wherein at least one list of rating criteria, requiring at least one input at least by a clinical personnel, is produced.

14. The method as claimed in claim 1, wherein at least one piece of contact information pertaining to the study is provided for the patient.

15. The method as claimed in claim 1, wherein at least one input step involves at least one input and/or output unit being used to query and/or input at least one further patient parameter.

16. The method as claimed in claim 1, wherein at least one input and/or output unit is used to make available at least one piece of suitability information of the patient to a clinical personnel associated with the patient.

17. The method as claimed in claim 1, wherein at least one patient parameter is captured in automated fashion and is processed in a computerized manner by means of at least one sensor unit, in particular a sensor unit of the input and/or output unit.

18. The method as claimed in claim 17, wherein at least one output parameter for an output of at least one piece of study information is adapted on the basis of the patient parameter captured by means of the sensor unit.

19. A patient recruitment system, in particular for recruiting patients for clinical studies, in particular configured to perform a method as claimed in claim 1, having at least one patient recruitment server that is configured to carry out at least one matching process for matching at least one patient parameter of at least one patient with at least one recruitment parameter of at least one study, and to carry out at least one rating process for generating at least one suitability parameter of a suitability of the patient for the study on the basis of the patient parameter and the recruitment parameter, wherein the patient recruitment server is configured to carry out at least one association process for making at least one association between the study and the patient on the basis of the suitability parameter.

20. The patient recruitment system as claimed in claim 19, further comprising at least one input and/or output unit, connectable to the patient recruitment server, that is configured for an input of at least one patient parameter and/or of a participation intention and/or for an output of at least one piece of study information.

21. The patient recruitment system as claimed in claim 20, wherein the input and/or output unit is configured for arrangement in at least one healthcare institution, in particular in at least one patient room and/or in at least one doctors' room and/or in at least one waiting room.

22. The patient recruitment system as claimed in claim 20, wherein the input and/or output unit comprises at least one display arranged in a patient room.

23. The patient recruitment system as claimed in claim 19, wherein the input and/or output unit comprises at least one mobile apparatus.

24. An input and/or output unit configured for use in a patient recruitment system as claimed in claim 19.

25. A use of a method as claimed in claim 1 for recruiting at least one patient for at least one study, in particular for a clinical study, advantageously for a phase III clinical study aimed for example at testing a therapeutic effect of an active substance and in particular the approval thereof.

26. The patient recruitment system as claimed in claim 1, wherein the matching step and/or the association step are performed repeatedly.
